(19) United States
(12) Patent Application Publication
(10) Pub. No.: US 2007/0135770 A1
(43) Pub. Date: Jun. 14, 2007

(54) Endoscopic device stabilizer

(75) Inventors: John V. Hunt, Cincinnati, OH (US); Michael S. Cropper, Edgewood, KY (US); Richard C. Smith, Milford, OH (US); Richard F. Schwemberger, Cincinnati, OH (US); John P. Measamer, Cincinnati, OH (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Ethicon Endo-Surgery, Inc.

(21) Appl. No.: 11/301,891

(22) Filed: Dec. 13, 2005

(51) Int. Cl. A61M 5/32

(52) U.S. Cl. 604/174

(57) ABSTRACT

An endoscopic device stabilizer for stabilizing an endoscopic device when inserted in a mouth of a patient. The stabilizer includes a body adapted for receipt within the mouth of a patient. The body has an opening sized and shaped for receiving the endoscopic device when the device is inserted in the mouth of the patient. The stabilizer also includes a clamp attached to the body selectively moveable between a locked position in which the clamp resists movement of the endoscopic device relative to the body and an unlocked position in which the clamp permits movement of the endoscopic device relative to the body.

### BACKGROUND OF THE INVENTION

The present invention relates generally to endoscopic equipment, and more particularly to a stabilizer for stabilizing an endoscopic device.

Endoscopic devices are medical devices used for examining and performing surgery inside a patient. Endoscopic devices typically include an elongate member for entering the body through a natural orifice (e.g., a patient's mouth, rectum, vagina or ureter) or an incision and for extending into or through a body canal (e.g., a patient's throat or colon), an internal cavity or an organ so the interior of the body can be visually examined or operated on without making large incisions. Thus, endoscopic devices permit less invasive examination and surgeries than conventional examination and surgical methods. Many endoscopic devices may be articulated or bent to follow body canals to a particular site of interest within the body. One type of endoscopic device is referred to as an endoscope or laparoscope, which allows a user to view interior areas of a patient's body to make visual examinations or to view the areas of the body where surgery is being performed. Other types of endoscopic devices include suturing equipment, scalpels, and forceps. An endoscopist guides the endoscopic device through the patient's body to position a functional end or tip of the device at the site of interest so the device can perform the task for which it is intended.

An endoscopist usually must use one or both hands to guide the endoscopic device into position. For example, when guiding an endoscopic device into a patient's stomach through his or her mouth, the endoscopist must use one hand adjacent the patient's mouth to guide the endoscope into the mouth and hold the endoscope in position. Frequently, the patient's throat muscles tend to move the endoscope involuntarily. Thus, the endoscopist must keep one hand on the device to hold it in position against the forces of the patient involuntary reflexes. Because the endoscopist's hand is needed to guide the device and hold it in position, the hand is not available for performing other tasks such as operating video recording equipment to record the views seen through the device or to operate other devices used to perform diagnosis, therapy or surgery. Similarly, when guiding an endoscopic device into a patient through an orifice other than a mouth (i.e., either a natural orifice or an incision), the endoscopist faces similar problems. Accordingly, there is a need for endoscopic equipment which will guide endoscopic devices into a patient and temporarily hold them in position within the patient.

In addition, even when an endoscopist uses one hand to guide endoscopic devices into a patient's mouth, there are occasions when the endoscopic equipment contacts the teeth of the patient, potentially damaging the patient's teeth or the endoscopic device. Thus, there is a need for a endoscopic equipment for preventing contact between the patient's teeth and the endoscopic device to eliminate a potential for damage to the patient's teeth and the endoscopic device.

### SUMMARY OF THE INVENTION

Briefly, the present invention includes an endoscopic device stabilizer for stabilizing an endoscopic device when inserted in a mouth of a patient. The stabilizer comprises a body adapted for receipt within the mouth of a patient. The body has an opening sized and shaped for receiving an endoscopic device when the device is inserted in the mouth of the patient. Further, the stabilizer comprises a clamp attached to the body selectively moveable between a locked position in which the clamp resists movement of the endoscopic device relative to the body and an unlocked position in which the clamp permits movement of the endoscopic device relative to the body.

In another aspect, the invention includes a method of inserting an endoscopic device into a mouth of a patient. The method comprises positioning a stabilizer into the mouth of the patient, inserting the endoscopic device into the stabilizer, and advancing the endoscopic device through the stabilizer.

In still another aspect, the invention includes a method of inserting an endoscopic device in an orifice in a patient. The method comprises positioning a stabilizer into the orifice of the patient, inserting the endoscopic device into the stabilizer, and advancing the endoscopic device through. the stabilizer.

Other features of the present invention will be in part apparent and in part pointed out hereinafter.

### BREIF DESCRIPTION OF THE DRAWINGS

FIG. 1 is a perspective of an endoscopic device stabilizer of a first embodiment of the present invention positioned in a patient's mouth;

FIG. 2 is a horizontal plan in partial section of the endoscopic device stabilizer of the first embodiment;

FIG. 3 is a horizontal plan in partial section similar to FIG. 2 but with a clamp of the stabilizer in a locking position; and

FIG. 4 is a perspective of an endoscopic device stabilizer of a second embodiment of the present invention.

FIG. 5 is a partially transparent perspective of an endoscopic device stabilizer of a third embodiment of the present invention.

FIG. 6 is a horizontal plan in partial section of an endoscopic device stabilizer of a fourth embodiment of the present invention.

FIG. 7 is a horizontal plan in partial section of an endoscopic device stabilizer of a fifth embodiment of the present invention.

FIG. 8 is a partially transparent perspective of an endoscopic device stabilizer of a sixth embodiment of the present invention.

FIG. 9 is a partially transparent perspective of an endoscopic device stabilizer of a seventh embodiment of the present invention.

FIG. 10 is a partially transparent perspective of an endoscopic device stabilizer of an eighth embodiment of the present invention.

FIG. 11 is a perspective of an endoscopic device stabilizer of a ninth embodiment of the present invention.

Corresponding reference characters indicate corresponding parts throughout the several views of the drawings.

### DETAILED DESCRIPTION OF THE PREFERRED EMBODIMENT

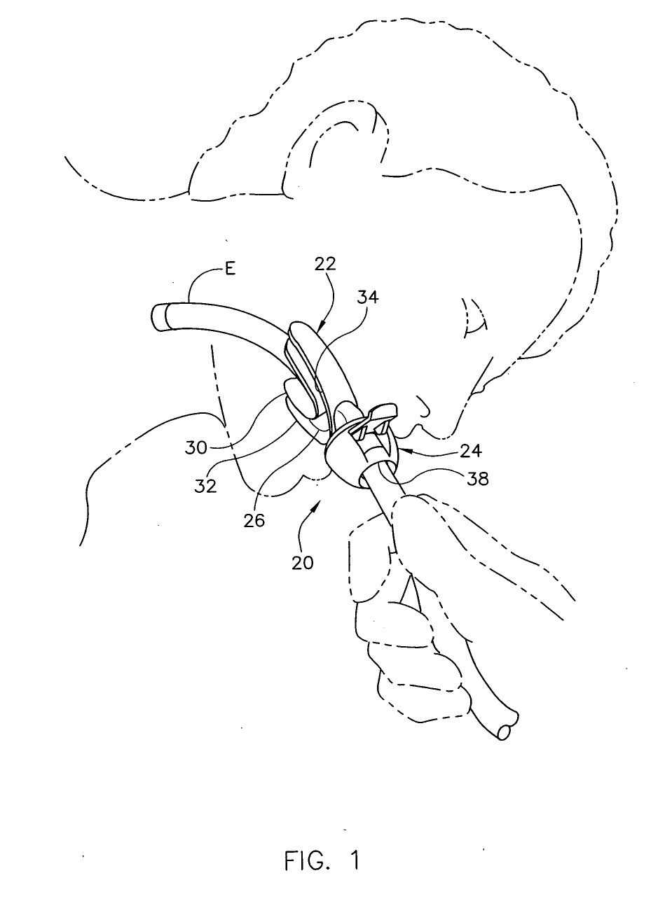

Referring now to the drawings and in particular to FIG. 1, an endoscopic device stabilizer of the present invention is designated in its entirety by the reference numeral 20. The stabilizer 20 is intended for use with an endoscopic device such as an endoscope E. The stabilizer 20 generally comprises a mouth-piece body, generally designated by 22, and a clamp, generally designated by 24.

The mouth-piece body 22 is adapted for receipt within the mouth of a patient as shown in FIG. 1. The body 22 is generally u-shaped for receipt between upper and lower teeth of the patient. The body 22 includes a plate or block 26 adapted for positioning between a patient's upper and lower teeth for preventing the upper teeth from contacting the lower teeth. In one embodiment, the plate 26 has rounded ends 28 for reducing a potential for injuring the patient's mouth. The plate 26 has an inner flange 30 extending outward from each face of the plate adjacent its inner edges. The inner flange 30 is adapted for positioning inside the patient's upper and lower teeth when the plate 26 is received in the patient's mouth between the upper and lower teeth. The plate 26 also has an outer flange 32 extending outward from each face of the plate adjacent its outer edges. The outer flange 32 is adapted for positioning outside the patient's upper and lower teeth when the plate is received between the upper and lower teeth of the patient. Although the plate 26 may have other thicknesses without departing from the scope of the present invention, in one embodiment the plate has a thickness greater than a width of the endoscopic device with which the stabilizer 20 is intended to be used. Although the inner and outer flanges 30, 32, respectively, may spaced by other distances without departing from the scope of the present invention, in one embodiment the inner and outer flanges are uniformly spaced along their lengths by a distance of between about two millimeters and about ten millimeters, thereby forming channels 34 for receiving the upper and lower teeth to hold the stabilizer in position in the patient's mouth. The flanges 30, 32 may having tapering widths and thickness, may have uniform widths and thicknesses, or may have other shapes without departing from the scope of the present invention. Moreover, the widths and thicknesses of the inner flange 30 may differ from those of the outer flange 32. Although the inner and outer flanges 30, 32, respectively, may have other overall widths without departing from the scope of the present invention, in one embodiment the inner and outer flanges have overall widths between about five millimeters and about ten millimeters. Although the inner and outer flanges 30, 32, respectively, may have other thicknesses without departing from the scope of the present invention, in one embodiment the inner and outer flanges have thicknesses of about two millimeters. Further, the edges and ends of the flanges 30, 32 may be rounded as shown to reduce a potential for injuring the patient's mouth. Those of ordinary skill in the art will appreciate that the overall size and shape of the body 22 may be modified to fit a variety of patient mouth sizes and shapes.

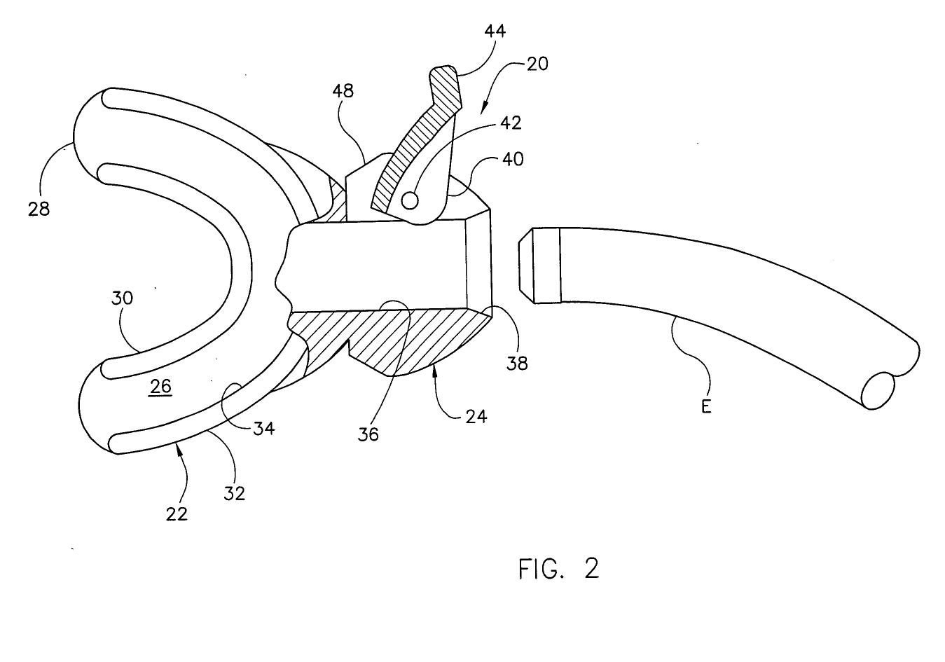

As illustrated in FIG. 2, the body includes an opening 36 sized and shaped for receiving the endoscopic device E when the device is inserted into the mouth of the patient. Although the opening 36 may have other shapes and sizes without departing from the scope of the present invention, in one embodiment the opening is fully enclosed and circular. Further, the opening 36 of this embodiment has a diameter between about one millimeter and about twenty millimeters. Further, the opening 36 may include a beveled edge 38 at one end to provide a wider entry port for easing insertion of the endoscopic device E into the opening. Although the body 22 may be made of other materials without departing from the scope of the present invention, in one embodiment the body is made from a rigid polymer such as polypropylene or ethyl methacrylate with a soft gel liner or a pliable polymer such as polyvinyl. It is envisioned that the opening 36 of the stabilizer 20 may also include a seal system (not shown) to reduce insufflation or gas loss. Further, the stabilizer 20 may have more than one opening 36 for accommodating additional endoscopic devices without departing from the scope of the present invention. In addition, it is envisioned the stabilizer 20 of the present invention may be used in combination with a conventional shape locking tube (not shown) to further stabilize the endoscopic device. Alternatively, it is envisioned a shape locking tube may be integrally formed with the stabilizer 20.

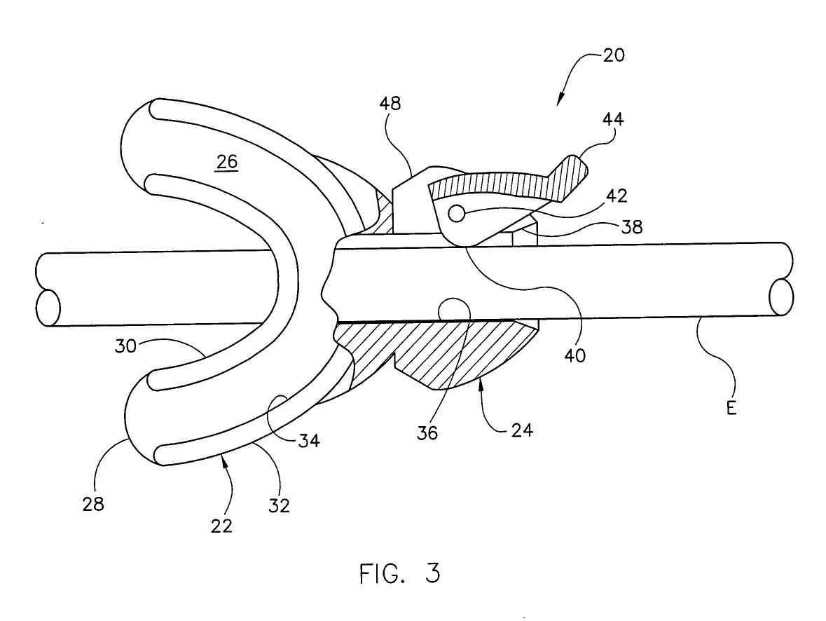

As further illustrated in FIG. 2, the clamp 24 in one embodiment comprises a cam 40 pivotally mounted on a pin 42 for selectively movement between an unlocked position as shown in FIG. 2 in which the clamp permits movement of the endoscopic device E in the opening 36 of the body 22 and a locked position as shown in FIG. 3 in which the clamp resists movement of the endoscopic device relative to the body. The clamp 24 also includes a lever 44 integrally formed with the cam 40 for applying leverage to the cam to pivot it between the locked position and the unlocked position. As will be appreciated by those skilled in the art, the clamp 24 may include a collar 48 on which the cam 40 is mounted. In one embodiment, this collar 48 is integrally formed with the body 22.

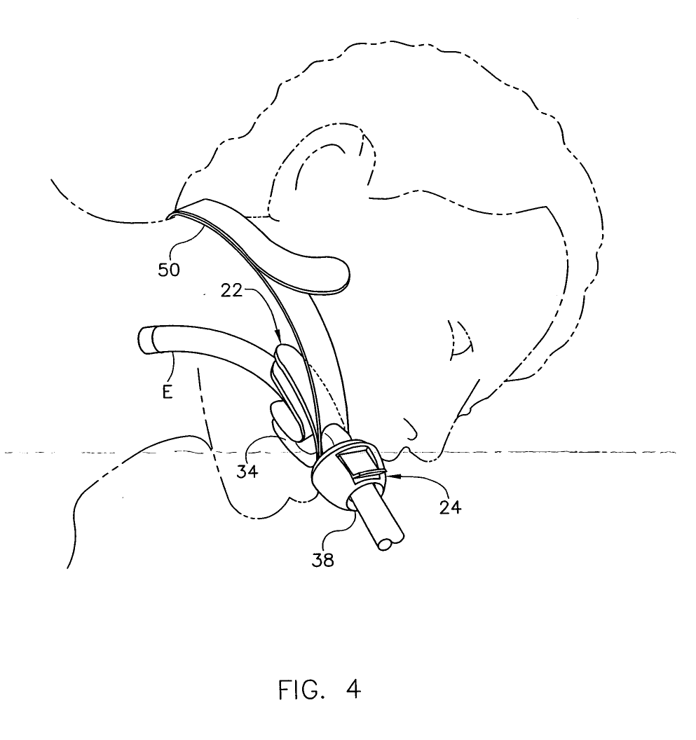

The stabilizer 20 of the present invention may be held in place in several different ways. For example, if the patient is awake and unsedated, the body may be held in place between the patient's upper and lower teeth as shown in FIG. 1. In other instances, the body 20 may be positioned between the patient's upper and lower teeth and a strap 50 may be used to hold the stabilizer 20 in place as shown in FIG. 4. The strap at least partially encircles the patient's head and may be fastened, such as with a gripper fastener, hook and loop fasteners (e.g., Velcro fasteners), tape strap, or with a buckle (not shown). It is also envisioned that the stabilizer 20 may be held in position with tape, adhesive, a rubber band, or ear loops. Alternatively, the plate 26 may be made of a pliable polymer such as polyvinyl material that softens when heated and bonds when cooled. To use this alternate embodiment, the plate 26 is heated before the stabilizer 20 is positioned in the patient's mouth. The patient bites the plate 26 and the plate is allowed to cool as the patient bites it so the plate temporarily bonds to the patient's teeth. Still further, the stabilizer 20 may be attached to the patient's bed without departing from the scope of the present invention.

The stabilizer 20 described above may be used to insert an endoscopic device (e.g., an endoscope) into a mouth of a patient. The endoscopist positions the stabilizer 20 in the mouth of the patient, inserts the endoscopic device E into the stabilizer opening 36, and advances the endoscopic device through the stabilizer. Once the endoscopic device E is in the a desired position with the tip of the endoscopic device in a desired position within the patient, the clamp 24 may be moved from the unlocked position to the locked position by rotating the lever 44 from the position shown in FIG. 2 to the position shown in FIG. 3. As the lever 44 is rotated, the cam 40 rotates about the pin 42 so it engages the surface of the endoscopic device to hold the endoscopic device relative to the stabilizer 20 so it stays in position in the patient. As described above, the stabilizer 20 may be attached to the patient prior to inserting the endoscopic device into the stabilizer by one of the methods described above.

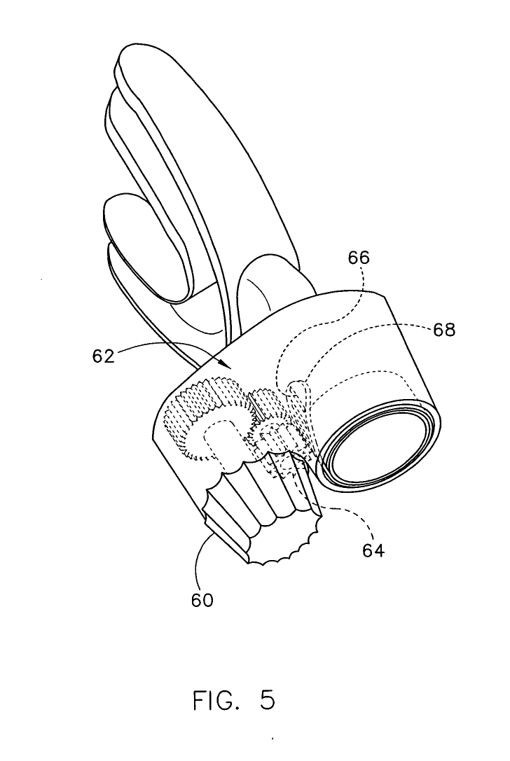

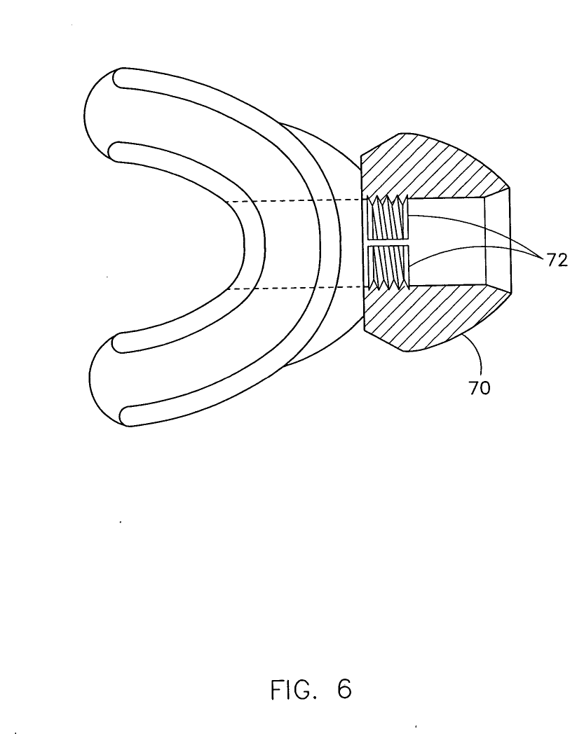

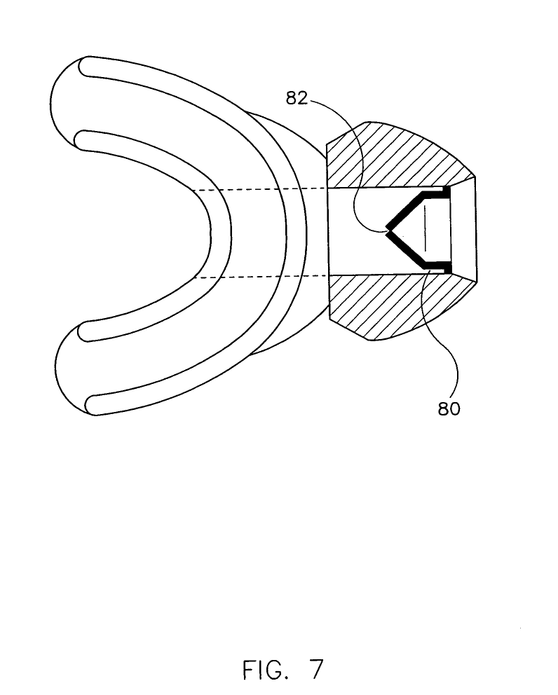

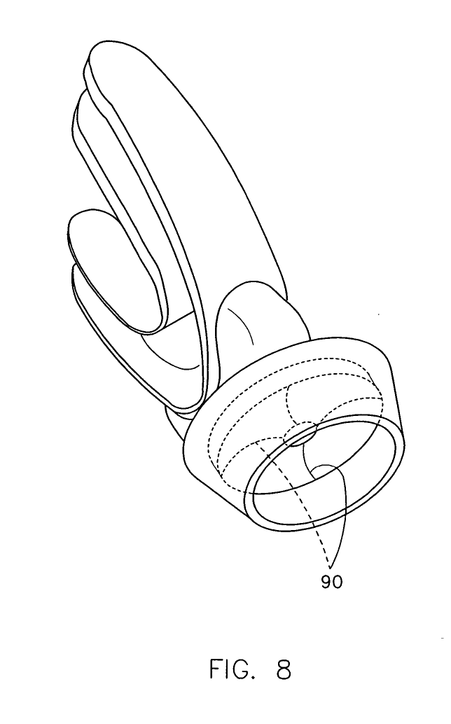

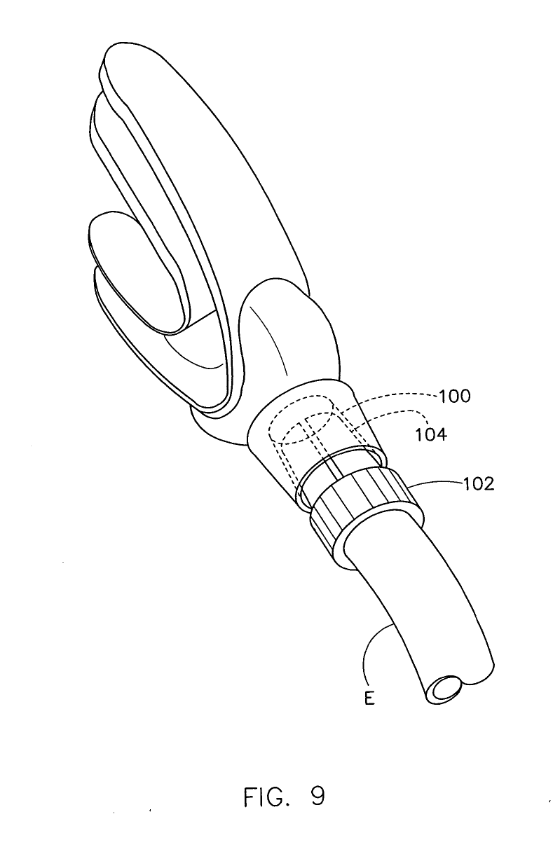

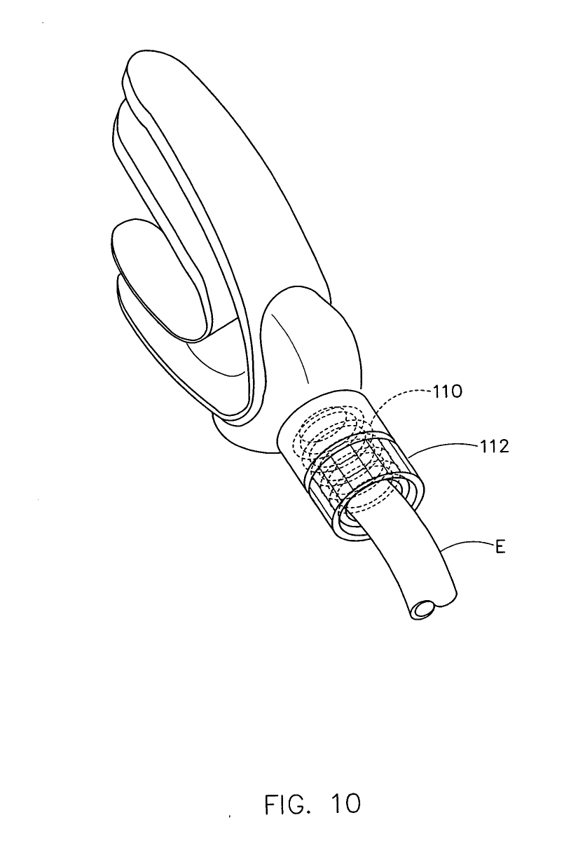

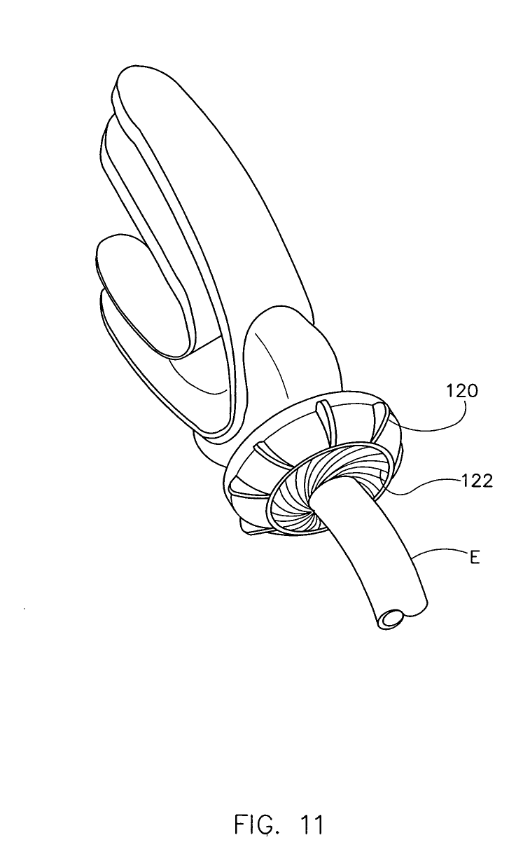

The clamp 24 may have other configurations without departing from the scope of the present invention. For example, the clamp 24 may have a strap clamp configuration as illustrated in FIG. 5, a collet configuration as illustrated in FIG. 6, a trocar-type configuration as illustrated in FIGS. 7 and 8, a rubber stopper configuration as illustrated in FIG. 9, a compressive sleeve lock configuration as illustrated in FIG. 10, an iris-type lock configuration as illustrated in FIG. 11, or a detent configuration without departing from the scope of the present invention. In the strap clamp configuration of FIG. 5, a knob 60 is provided for actuating the clamp. The knob 60 is operatively connected to a train of gears, generally designated 62, that drives a pinion 64. The pinion engages a rack 66 formed by a series of slots in a strap 68. The strap 68 is selectively tightened or loosened around the endoscopic device E (not shown) by turning the knob 60. In the collet configuration of FIG. 6, an internally threaded nut 70 engages fingers 72 so that the fingers are compressed against the endoscopic device E (not shown) when the nut is turned in one direction (i.e., clockwise) and released when the nut is turned in an opposite direction. In the trocar-type configuration shown in FIG. 7, a flexible duck bill seal 80 is formed for receiving the endoscopic device E (not shown). The seal 80 has a slit 82 which opens as the seal deforms to receive the endoscopic device E. In the configuration shown in FIG. 8, a plurality of flexible leaves 90 are provided around the opening. The leaves 90 deform to receive the endoscopic device E. In the stopper configuration shown in FIG. 9, the opening 36 includes a tapered portion 100. A stopper 102 having fingers 104 is received within the tapered portion 100. When the stopper 102 is pushed into the tapered portion 100 of the opening 36, the fingers 104 grip the endoscopic device E. In the compressive sleeve lock configuration illustrated in FIG. 10, an internal spring 110 grasps the endoscopic device E to hold it in position. The force applied by the spring 110 on the endoscopic device E may be adjusted by turning a knob 112 attached to one end of the spring. When the knob 112 is turned in one direction, an inner diameter of the spring is reduced thereby increasing the force applied by the spring, and when the knob is turn in an opposite direction, the inner diameter of the spring is increased thereby decreasing the force applied by the spring. In the iris configuration shown in FIG. 11, the user turns a collar 120 surrounding an iris 122 to open and close the iris around the endoscopic device E. Because each of these clamp configurations is generally conventional, they will not be described in further detail. As will be appreciated by those skilled in the art, various changes and modifications may be made to the configurations described above without departing from the scope of the present invention.

As will also be appreciated by those skilled in the art, the stabilizer 20 of the present invention, stabilizes the endoscopic device E to resist movement during therapy, diagnosis or surgery. With the endoscopic device E stabilized by the stabilizer 20 of the present invention, the endoscopist's hands are free to perform other tasks such as approximating tissue, suturing, or cutting tissue. Therefore, those skilled in the art will appreciate that the stabilizer 20 of the present invention has several advantages over prior art devices.

In one embodiment, the stabilizer device described above may be used to perform intra-abdominal surgery. The stabilizer device is inserted in an orifice in the patient and secured relative to the patient once the device is in the proper position. The endoscopic device is inserted in the stabilizer device and advanced through the stabilizer device so the endoscopic device extends into an internal cavity in the patient such as an abdominal cavity or a thoracic cavity. An incising instrument is inserted through the endoscopic device, advanced to a desired location within the cavity and an incision is made in an internal wall of the cavity. The endoscopic device may be locked in position relative to the stabilizer device to permit inspection of the cavity and permit a surgical procedure to be performed within the cavity. The endoscopic device may be repositioned or removed by unlocking the stabilizer device. When the procedure is complete, the stabilizer may be unlocked and all the endoscopic devices and instruments may be removed. The orifice mentioned above in this intra-abdominal surgery procedure may be a natural orifice such as a mouth of the patient.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there inay be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. An endoscopic device stabilizer for stabilizing an endoscopic device when inserted in a mouth of a patient, said stabilizer comprising:
a body adapted for receipt within the mouth of a patient, said body having an opening sized and shaped for receiving the endoscopic device when said device is inserted in the mouth of the patient; and
a clamp attached to the body selectively moveable between a locked position in which the clamp resists movement of the endoscopic device relative to the body and an unlocked position in which the clamp permits movement of the endoscopic device relative to the body.

2. An endoscopic device stabilizer as set forth in claim 1 wherein said body is generally shaped for receipt between upper and lower teeth of the patient.

3. An endoscopic device stabilizer as set forth in claim 2 wherein at least a portion of said body is generally U-shaped.

4. An endoscopic device stabilizer as set forth in claim 3 wherein said U-shaped portion of the body has in inner flange adapted for positioning inside the patient's upper and lower teeth when the body is received between the upper and lower teeth of the patient and an outer flange adapted for positioning outside the patient's upper and lower teeth when the body is received between the upper and lower teeth of the patient.

5. An endoscopic device stabilizer as set forth in claim 2 wherein the body has a thickness greater than a width of the endoscopic device.

6. An endoscopic device stabilizer as set forth in claim 1 wherein the opening in the body is circular.

7. An endoscopic device stabilizer as set forth in claim 2 wherein the body is adapted for bonding to the patient's upper and lower teeth when the body is received between the upper and lower teeth of the patient.

8. An endoscopic device stabilizer as set forth in claim 2 wherein the clamp comprises a cam lever.

9. An endoscopic device stabilizer as set forth in claim 1 further comprising a strap extending from the body for attaching the stabilizer to the patient.

10. An endoscopic device stabilizer as set forth in claim 9 wherein the strap at least partially encircles a head of the patient when attaching the stabilizer to the patient.

11. A method of inserting an endoscopic device into a mouth of a patient, said method comprising:
positioning a stabilizer into the mouth of the patient;
inserting the endoscopic device into the stabilizer; and
advancing the endoscopic device through the stabilizer.

12. A method as set forth in claim 11 further comprising attaching the stabilizer to the patient.

13. A method as set forth in claim 12 wherein the attaching step is performed by strapping the stabilizer to the patient.

14. A method as set forth in claim 13 further comprising clamping the endoscopic device to the stabilizer once a tip of the device reaches a desired position within the patient.

15. A method of inserting an endoscopic device into an orifice in a patient, said method comprising:
positioning a stabilizer in the orifice of the patient;
inserting the endoscopic device into the stabilizer; and
advancing the endoscopic device through the stabilizer.

16. A method as set forth in claim 15 wherein the orifice is a natural orifice.

17. A method as set forth in claim 16 wherein the orifice is a mouth of a patient.

18. An endoscopic device stabilizer for stabilizing an endoscopic device when inserted in a mouth of a patient, said stabilizer comprising:
means for receiving the endoscopic device; and
means for selectively resisting movement of the endoscopic device relative to the receiving means.
